(19) United States
(12) Patent Application Publication
(10) Pub. No.: US 2023/0011750 A1
(43) Pub. Date: Jan. 12, 2023

(54) BREATHABLE COOLING PILLOW AND METHOD OF MAKING SAME

(71) Applicant: Standard Fiber, LLC, Burlingame, CA (US)

(72) Inventors: Chun Leung Chan, San Francisco, CA (US); Yongxing Lin, Shanghai City (CN); Yongjun Li, Shanghai City (CN)

(21) Appl. No.: 17/861,782

(22) Filed: Jul. 11, 2022

Related U.S. Application Data
(60) Provisional application No. 63/220,851, filed on Jul. 12, 2021.

(51) Int. Cl. A47G 9/10; A61F 7/02

(52) U.S. Cl. CPC A47G 9/1036; A61F 7/02; A61F 2007/0219; A61F 2007/0244; A47G 2009/1018

(57) ABSTRACT

A breathable cooling pillow includes a polymeric foam layer having a top surface, a bottom surface, and sides extending between the top surface and the bottom surface and a polymeric gel cooling layer disposed directly on the top or bottom surface. The gel cooling layer is molded, at least in part, into the foam layer. A method of making a breathable cooling pillow is disclosed.

### BACKGROUND

This application relates to cooling pillows and methods of making same, and more particularly to breathable cooling pillows and methods of making breathable cooling pillows. Cooling pillows are known. Some pillows include shredded foam, such as memory foam that is infused with a gel or copper to draw heat from the user's body. Other cooling pillows may have a buckwheat hull filler which creates air pockets and prevents heat retention.

Still other cooling pillows include specific fabric shells that have cooling characteristics. Bamboo, for example, is a moisture-wicking material that provides a cooling effect. Other fabrics may have phase-change materials that absorb body heat and regulate temperature by changing phase, for example from solid to liquid.

Other known cooling pillows 1 include a pad 2 filled with a cooling gel (see, FIG. 1). The pad 2 is adhered to an underlying pillow, such as a foam material. These cooling gels provide substantial cooling characteristics which can be considerably greater than prior known cooling pillow materials. Such pads are formed from membranes sealed to one another to form cells that contains the cooling gel. Often such pads are formed with a matrix of cells, each of which includes a volume of cooling gel. The gel is typically a viscous liquid.

While such gel pads function well to provide cooling, they have their drawbacks. For example, if a cell is broken or pierced, the gel will leak from the pad. This results in a loss of cooling ability. And while such a pad may have many cells, and the loss of one cell may not adversely affect the cooling ability, the loss of a number of cells can result in a substantial loss of cooling ability. In addition, gel leaking from a pierced or broken cell is messy and must be cleaned well in order to the continue using the pillow. Moreover, the gel can leak into other materials, such as the underlying foam pillow creating a greater mess.

Further, the manufacture of such gel pad pillows requires a number of steps to assure the proper configuration of the final product. For example, in one known manufacturing method, a gel pad is positioned in a mold. A sheet or membrane 3 (see, FIG. 1) is positioned on the pad, and a polymer formulation is applied onto the sheet or membrane. The mold is then closed and heat is applied to (and in some methods a gas such as carbon dioxide or water vapor, or warm or hot water is injected into) the mold to foam the polymer formulation and form the gel pad pillow. The sheet or membrane is needed to prevent the polymer formulation from seeping under the gel pad in the mold.

One drawback to this method is that it requires a number of steps including forming the gel pad, applying and adhering the sheet or membrane to the pad, properly positioning the pad and sheet in the mold so that the polymer doesn't seep under pad, applying the polymer formulation and molding/forming (e.g., foaming) the pillow.

In addition, because the sheet or membrane is intended to prevent seepage of the pillow forming polymer formulation, in addition to being impermeable to the pillow forming polymer formulation, it is also impermeable to air. That is, the sheet or membrane prevents air flow into and out of the pillow. As such, while the pillow may be a cooling pillow, it is not breathable.

In order to make such gel pad pillows breathable, holes must be made through the sheet or membrane and the gel pad, and into the foamed pillow. In making the holes through the gel pad, there is a possibility of puncturing the gel pad, which can result in leakage of the gel, loss of cooling ability and mess.

Accordingly, there is a need for a gel-type cooling pillow that is breathable. Desirably, such a pillow uses a cooling formulation that forms a sheet that provides the cooling capability of a liquid gel in a solid gel form. More desirably still, such a pillow is breathable without compromising the integrity of the gel or the cooling capability of gel and pillow. Still more desirably, a method for making the pillow is less complex (requires less manufacturing steps), requires less materials and provides a high level of confidence of pillow quality.

### SUMMARY

A breathable cooling pillow includes a polymeric foam layer having a top surface, a bottom surface, and sides extending between the top surface and the bottom surface. A polymeric gel cooling layer is disposed directly on the top or bottom surface. The gel cooling layer is molded, at least in part, into the foam layer.

In embodiments, the polymeric foam layer is a foamed polyurethane. The polymeric foam layer can be a polyurethane memory foam. The gel cooling layer can be, for example, a non-foamed polyurethane. In embodiments, the gel cooling layer extends fully across the top or bottom surface to the sides of the pillow.

In embodiments, holes are formed in the pillow. The holes can be formed in the gel cooling layer and at least in part into the foam layer. In embodiments, the holes in the foam layer extend fully through the foam layer.

A method for making a breathable cooling pillow includes the steps of applying a gel cooling formulation on a bottom of a mold out to the sides of the mold and partially curing the gel formulation to form a partially cured cooling gel layer. The method includes applying a foamable polymeric formulation directly to the partially cured cooling gel layer and foaming the foamable polymeric formulation to form a foamed layer.

The method further includes curing the cooling gel layer to form the breathable cooling pillow and removing the breathable cooling pillow from the mold.

In methods, the gel cooling layer is a non-foamed polyurethane. In methods, the foamable polymeric layer is polyurethane, and can be a memory foam formulation.

Methods can further include forming holes in the breathable cooling pillow. The holes can be formed in the gel cooling layer and at least in part into the foam layer. The holes in the foam layer can extend fully through the foam layer.

Other features, and advantages of the disclosure will be apparent from the following description, taken in conjunction with the accompanying sheets of drawings, wherein like numerals refer to like parts, elements, components, steps, and processes.

### DESCRIPTION OF THE DRAWINGS

FIG. 1 is a photograph of a breathable cooling pillow in accordance with an embodiment, the pillow being cut and being shown on top of a known cooling gel pillow;

FIGS. 2A and 2B are photographs of the cooling gel being applied to a mold (FIG. 2A) and fully applied to the mold (FIG. 2B); and

FIG. 3 is a photograph of the polymer pillow formulation being applied to the cooling gel in the mold, and prior to forming (foaming) the pillow.

### DETAILED DESCRIPTION

While the present disclosure is susceptible of embodiment in various forms, there is shown in the drawings and will hereinafter be described one or more embodiments with the understanding that the present disclosure is to be considered illustrative only and is not intended to limit the disclosure to any specific embodiment described or illustrated.

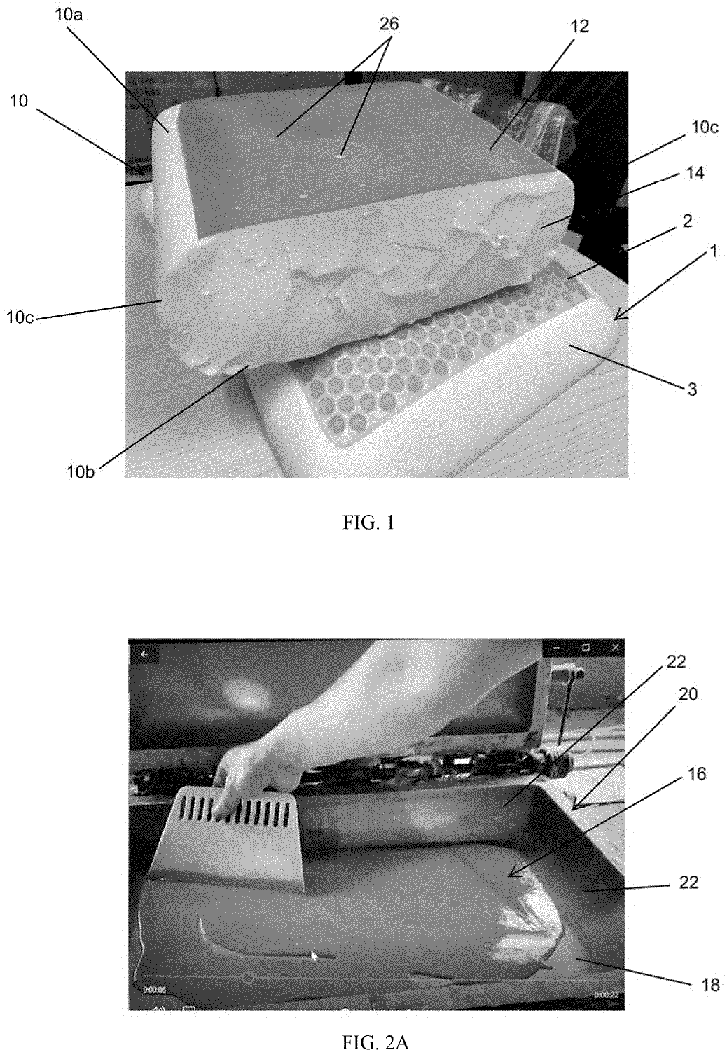

Referring now to the figures and in particular to FIG. 1 there is shown an embodiment of a breathable cooling pillow 10 in accordance with the present disclosure. The pillow 10 has a top surface 10a, a bottom surface 10b and sides 10c. The pillow 10 includes a solidified gel cooling layer 12 on the top surface 10a and a foam layer 14. The solidified gel layer 12 is a soft, flexible solidified gel that conforms to the contours of, for example, a user's head. The foam layer 14 is a foamed polymer formulation. The foam can be, for example, a conventional foamed polymer.

In an embodiment, the solidified gel layer 12 is a non-foamed polyurethane, and may be, for example, an elastomer. The polyurethane may include additional materials or additives for elasticity/flexibility, stability, color, mold slip and the like. Other additives will be understood by those skilled in the art.

The foam layer 14 can also be a polyurethane, such as a foamed polyurethane. The polyurethane can be formed into a foam by the addition of a gas, water vapor, warm or hot water during the manufacturing or molding process. The polyurethane foam may be a memory foam and may be formed in an open cell or a closed cell structure.

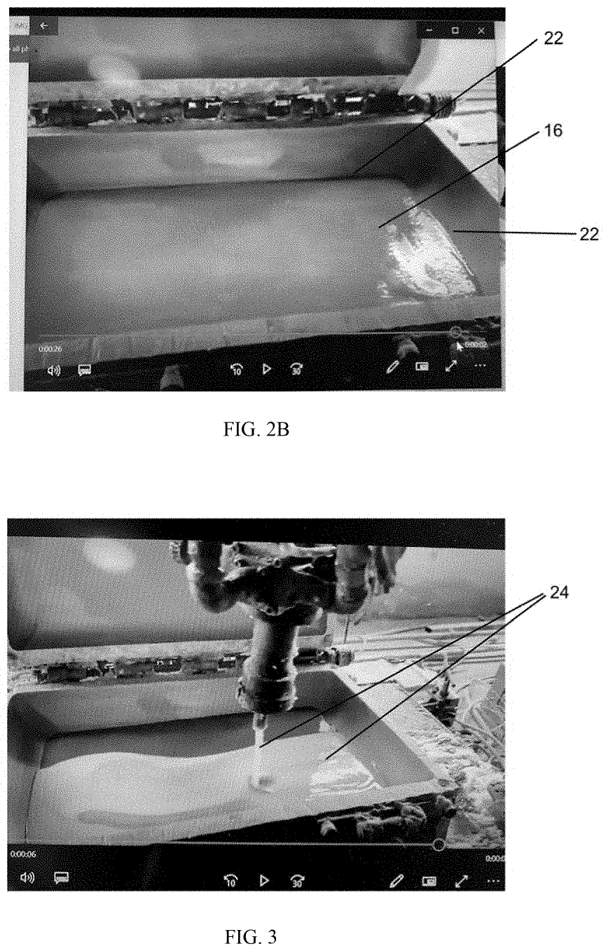

Referring now to FIGS. 2-4, in the manufacture of the pillow, a liquefied formulation 16 of the gel layer 12 is first applied to the bottom 18 of a pillow mold 20 as a liquid. The liquefied formulation 16 of the gel layer 12 is spread across the bottom 18 of the mold 20 out to the sides 22 of the mold 20 as seen in FIG. 3. At this point, depending upon the formulation of the gel layer, the liquefied formulation 16 of the gel layer 12 may be allowed to cure for a period of time to partially set. As such, in some methods the gel layer 12 is allowed to partially set.

The formulation 24 for the foam layer 14 is then applied over the partially cured or set gel layer 12. The mold 20 is closed and is heated to a temperature of about 95° F. to about 130° F. (about 35° C. to about 55° C.) for a period of about 5 to 10 minutes, and an agent to promote foaming such as a gas or warm/hot water is introduced into the mold 20. This is sufficient for the foam formulation 24 to expand to the interior volume of the mold 20 and to set and form the breathable cooling pillow 10.

The pillow 10 is then removed from the mold 20. In embodiments, holes 26 are then formed in the pillow 10, through the gel layer 12 and partially or fully through the foam layer 14. The holes 26 can be formed randomly or in a desired pattern.

It will be appreciated that there are numerous advantages of the present breathable cooling pillow over known pillow constructions. First, the gel layer 12 or cooling gel layer is solid. As such, there is no liquid to leak from individual cells. In addition, since no membrane is needed between a liquid gel pad layer and the foam pillow as in known pillows, the present gel layer 12 can be formed fully to the sides 10a,b,c of the pillow 10 (other than the sides of the pillow that result from expansion due to foaming). Moreover, because there is no membrane, the breathability of the pillow 10 is not reduced. And, holes 26 can be formed anywhere in the pillow 10, through the gel layer 12, without adversely impacting the integrity of the pillow 10 or the cooling capabilities of the pillow 10.

It will be appreciated by those skilled in the art that the relative directional terms such as top, bottom, sides, front, rear and the like are for explanatory purposes only and are not intended to limit the scope of the disclosure. All patents referred to herein, are hereby incorporated herein by reference, whether or not specifically done so within the text of this disclosure.

In the present disclosure, the words “a” or “an” are to be taken to include both the singular and the plural. Conversely, any reference to plural items shall, where appropriate, include the singular.

From the foregoing, it should also be understood that various changes and modifications to the presently disclosed embodiments will be apparent to those skilled in the art. Such changes and modifications can be made without departing from the spirit and scope of the present disclosure and without diminishing its intended advantages. It is therefore intended that such changes and modifications be covered by the appended claims.

1. A breathable cooling pillow, comprising:
a polymeric foam layer having a top surface, a bottom surface, and sides extending between the top surface and the bottom surface; and
a polymeric gel cooling layer disposed directly on the top or bottom surface, wherein the gel cooling layer is molded, at least in part, into the foam layer.

2. The breathable cooling pillow of claim 1, wherein the polymeric foam layer is a foamed polyurethane.

3. The breathable cooling pillow of claim 1, wherein the polymeric foam layer is a memory foam.

4. The breathable cooling pillow of claim 1, wherein the gel cooling layer is a non-foamed polyurethane.

5. The breathable cooling pillow of claim 1, further including holes formed in the pillow.

6. The breathable cooling pillow of claim 5, wherein the holes are formed in the gel cooling layer and at least in part into the foam layer.

7. The breathable cooling pillow of claim 6, wherein the holes in the foam layer extend fully through the foam layer.

8. The breathable cooling pillow of claim 1, wherein the gel cooling layer extends fully across the top or bottom surface to the sides.

9. A method for making a breathable cooling pillow, comprising:
applying a liquefied gel cooling formulation on a bottom of a mold out to sides of the mold;
partially curing the liquefied gel formulation to form a partially cured cooling gel layer;
applying a foamable polymeric formulation directly to the partially cured cooling gel layer;
foaming the foamable polymeric formulation to form a foamed layer;
curing the cooling gel layer to form the breathable cooling pillow; and
removing the breathable cooling pillow from the mold.

10. The method of claim 9, wherein the gel cooling layer is a non-foamed polyurethane.

11. The method of claim 9, wherein the foamable polymeric layer is polyurethane.

12. The method of claim 9, wherein the foamable polymeric layer is a memory foam formulation.

13. The method of claim 9, further including forming holes in the breathable cooling pillow.

14. The method of claim 13, wherein the holes are formed in the gel cooling layer and at least in part into the foam layer.

15. The method of claim 14, wherein the holes in the foam layer extend fully through the foam layer.
